(19) United States
(12) Patent Application Publication
(10) Pub. No.: US 2025/0375208 A1
(43) Pub. Date: Dec. 11, 2025

(54) PEDICLE SCREW IMPLANTATION GUIDE PLATE

(71) Applicant: National Defense Medical Center, Taipei (TW)

(72) Inventors: Wei-Hsiu Liu, Taipei (TW); Chih-Ming Huang, Taipei (TW); Wei-Ting Liu, Taipei (TW); Yi-Jia Lee, Taipei (TW)

(21) Appl. No.: 18/736,915

(22) Filed: Jun. 7, 2024

(51) Int. Cl. A61B 17/90; A61B 17/00; A61B 17/17

(52) U.S. Cl. CPC A61B 17/1757; A61B 17/1796; A61B 2017/00526

(57) ABSTRACT

A pedicle screw implantation guide plate, which comprises a guide body, a plurality of positioning holes, a longitudinal connection channel and an angle mark. The guide body has a first guide plate, a second guide plate and a connection part. The first guide plate is embedded in the second guide plate through the connection part. The plurality of positioning holes is disposed on the first guide plate and the second guide plate for inserting and fixing Kirschner wires. The longitudinal connection channel is disposed inside the first guide plate and the second guide plate for inserting the Kirschner wires to connect the first guide plate and the second guide plate, and to locate spinous process and transverse pedicles. The angle mark is disposed on the guide body, as a visual reference point to adjust a position of the guide body.

### BACKGROUND

### Technical Field

The present disclosure relates to a pedicle screw implantation guide plate, and in particular to a pedicle screw implantation guide plate used to assist spinal surgery.

### Description of Related Art

Spinal diseases refer to a type of disease that affects the structure and function of the spine and may have a significant impact on the patient's quality of life and body functions.

Here are some common spinal diseases:
1. Spondylolisthesis: Spondylolisthesis refers to the forward or backward movement of one vertebral body relative to the next vertebral body. This can be caused by fractures, disc degeneration, or other structural problems. Symptoms may include back pain, nerve compression, and radiating pain in legs.
2. Scoliosis: Scoliosis refers to the lateral curvature of the spine in a three-dimensional space. This may be congenital or occur during growth. Mild scoliosis may cause no symptoms, but severe cases may cause back deformation and organ compression.
3. Intervertebral Disc Herniation: Intervertebral disc herniation occurs when the outer annulus fibrosus of the intervertebral disc is torn and the inner softcore herniations. It may cause nerve root compression, back and neck pain, radiating pain, numbness, and muscle weakness.
4. Vertebral Fracture: Vertebral fracture may be caused by trauma, compression, osteoporosis, or other factors. It may cause vertebral collapse or compression fracture. Fractures may cause severe pain and require surgery to stabilize the vertebral body.
5. Spinal Tumor: Spinal Tumor refers to the abnormal cell proliferation that forms in or around the spine. Tumors may be primary (originating from spinal tissue) or metastatic (from other sites).

However, the current main treatment method is surgical intervention and internal fixation with screws implantation. During the operation, the surgical team first needs to push aside the patient's back muscles, fascia, etc., until the thoracic and lumbar vertebrae to be operated on are exposed. Then, drill holes and use Kirschner wires to locate the pedicle. Then, according to the surgical plan, the surgeon precisely implants the screws into the vertebrae to achieve stability and fixation. During the process, a fluoroscopic X-ray (C-arm) machine must be used several times to confirm whether the insertion route will damage the spinal cord. This may result in increased surgical time and excessive ionizing radiation, posing risks to the medical team and patient.

Accordingly, how to develop a pedicle screw implantation guide plate that can solve the above problems has become a problem to be solved in the technical field.

### SUMMARY

According to an embodiment of this disclosure, the present invention is directed to a pedicle screw implantation guide plate, which comprises a guide plate body, a plurality of positioning holes, a longitudinal connection channel, and an angle mark.

The guide plate body with an opposite pair of long side surfaces, an opposite pair of short side surfaces and an opposite pair of flat surfaces, wherein the guide plate body comprises a first guide plate and a second guide plate, the first guide plate has a connection part protruding from the first guide plate. The first guide plate is embedded in the second guide plate through the connection part along a direction that parallels to the pair of long side surfaces to form the guide plate body. The longitudinal connection channel is passed through an interior of the guide plate body which adjacent to one of the pair of long side surfaces to passe through the first guide plate and the second guide plate, the longitudinal connection channel is used to insert a first Kirschner wire to connect the first guide plate and the second guide plate. The plurality of positioning holes is passed through the pair of flat surfaces, the plurality of positioning holes is used to insert a plurality of second Kirschner wires. The angle mark is disposed on the pair of long side surfaces and the pair of short side surfaces of the guide plate body for serving as a visual reference point to adjust a position of the guide plate body.

According to yet another embodiment of this disclosure, the plurality of positioning holes is arranged in an array.

According to yet another embodiment of this disclosure, the plurality of positioning holes comprises a plurality of spinous process positioning holes, a plurality of pedicle positioning holes and a plurality of positioning holes for an upper edge of the vertebral body. The plurality of spinous process positioning holes is disposed in a central area of the guide plate body for inserting a part of the second Kirschner wire to drive into the spinous process in an individual body, wherein the central area comprises an overlapping portion of the connection part and the second guide plate and a portion of the first guide plate adjacent to the connection part. The plurality of pedicle positioning holes is disposed on the first guide plate and the second guide plate at both sides of the central area of the guide plate body for inserting another part of the second Kirschner wire to locate the pedicles in the individual body. The plurality of positioning holes for an upper edge of the vertebral body is located at both ends of the longitudinal connection channel to allow the first Kirschner wire to pass through the longitudinal connection channel and position an upper edge of the vertebral body on a body surface of the individual body.

According to yet another embodiment of this disclosure, the pedicle positioning hole has a medial inclination angle of 4° to 20°.

According to yet another embodiment of this disclosure, the spinous process positioning hole has a caudal inclination angle of 8° to 10° and a medial inclination angle of 5° to 7°.

According to yet another embodiment of this disclosure, the first guide plate and the second guide plate are integrally formed by additive manufacturing.

In summary, benefits of the present invention are: (1) The pedicle screw implantation guide plate can be customized for patients. (2) The direction of the Kirschner wire and the screw can be planned before surgery. (3) Reduce the increase in operation time and the generation of excessive ionizing radiation due to repeated use of fluoroscopic X-ray (C-arm) during the operation to confirm the insertion route.

The foregoing presents a simplified summary of the disclosure in order to provide a basic understanding to the reader. This summary is not an extensive overview of the disclosure, and it does not identify key/critical elements of the present invention or delineate the scope of the present invention. Its sole purpose is to present some concepts disclosed herein in a simplified form as a prelude to the more detailed description that is presented later. Many of the attendant features will be more readily appreciated as the same becomes better understood by reference to the following detailed description considered in connection with the accompanying drawings.

### BRIEF DESCRIPTION OF THE DRAWINGS

FIG. 1 is a stereogram of a pedicle screw implantation guide plate according to one embodiment of the present disclosure.

FIG. 2 is a stereogram of using a pedicle screw implantation guide plate according to one embodiment of the present disclosure.

FIG. 3 is perspective view of using a pedicle screw implantation guide plate according to one embodiment of the present disclosure.

### DETAILED DESCRIPTION

Please refer to embodiments of the present invention, which are illustrated in the accompanying drawings. Wherever possible, the same reference signs are used in the drawings and description to refer to the same or similar parts. In addition, the embodiment is only one of the design concepts of the present invention, and the following embodiments are not intended to limit the present invention.

To describe each embodiment of the present invention in more detail, the following description is supplemented by the accompanying drawings. It should be understood that, when an element is referred to as being “connected” or “disposed” to another element, it can mean that the element is directly located on another element, or that intervening elements may also be present to connect the element to another element through the intervening elements. In contrast, when an element is referred to as being “directly on” or “directly connected to” another element, it will be understood that there are no intervening elements present.

In addition, the terms “first”, “second” and “third” are only used to distinguish one element, component, region or section from another element, component, region, layer, or section. Rather than indicating its inevitable sequence. In addition, relative terms, such as “lower” and “upper,” may be used herein to describe the relationship of one element to another element, and it will be understood that these relative terms are intended to encompass different orientation of the device except from the orientation illustrated in the figures. For example, if the device in one of the figures is turned over, elements described as “below” other elements would then be oriented “above” the other elements. This only represents a relative orientation relationship, not an absolute orientation relationship.

The pedicle screw implantation guide plate 100 of the present invention is a customized guide plate made through additive manufacturing technology and is designed to provide assistance for spinal disease surgeries. The guide plate is manufactured by using advanced additive manufacturing technology and can be customized according to the patient's specific anatomical characteristics and surgical needs. At present, the mainstream surgical methods usually involve the pedicle screw implantation guide plate 100 which not only provides a higher degree of personalization, but also helps to improve the accuracy and safety of the operation. By planning the insertion direction before surgery, surgeon can more accurately determine the position of the pedicle screws. They no longer need to repeatedly use the C-arm to confirm the position during the surgery, which greatly simplifies the surgical process. In traditional surgery, the frequent use of C-arm may lead to an increase in operation time and increase the risk of radiation exposure to patients and medical teams.

To describe each embodiment of the present invention in more detail, the following description is supplemented by the accompanying drawings.

Additive Manufacturing (AM), also known as three-dimensional printing or rapid prototyping, is a technology for manufacturing objects that builds the three-dimensional structure of the object by adding materials layer by layer. Compared with traditional medical materials, medical materials produced by additive manufacturing technology are better in terms of adhesion to human bones, fixation, and tissue regeneration efficiency.

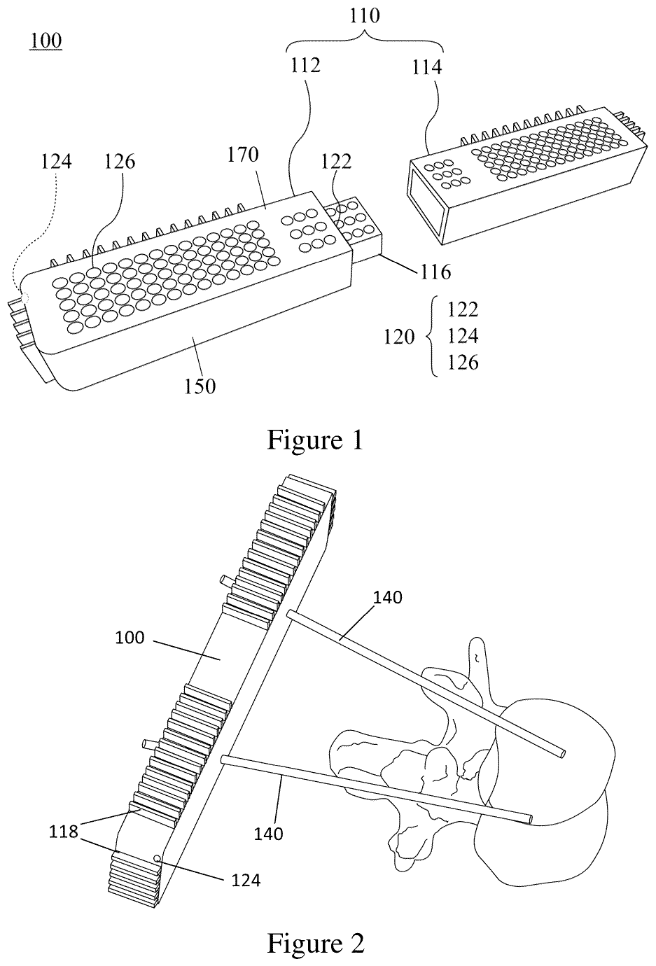

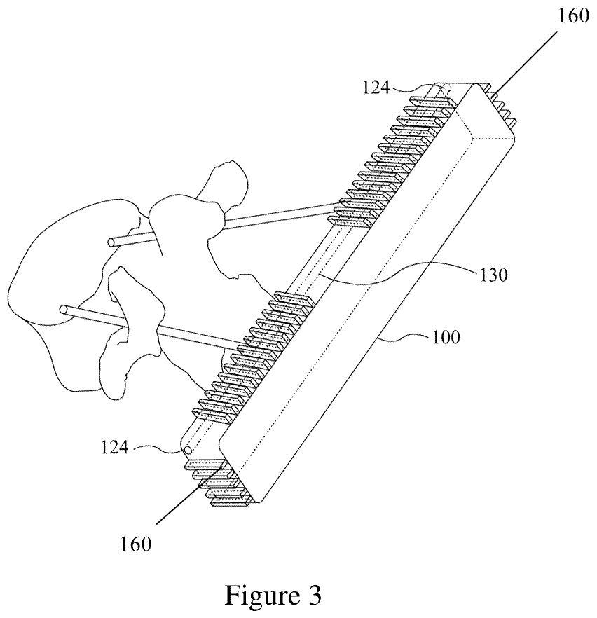

Please refer to FIGS. 1 to 3. According to an embodiment, the pedicle screw implantation guide plate 100 of the present invention has a guide plate body 110 made by additive manufacturing technology. The guide plate body 110 can be made of any of the following materials, but not limited thereto, that are compatible with human tissue, such as polylactic acid (PLA), polycaprolactone (PCL) or polyetheretherketone (PEEK), etc.

The guide plate body 110 has an opposite pair of long side surfaces 150, an opposite pair of short side surfaces 160 and an opposite pair of flat surfaces 170. Along the direction of the pair of long side surfaces 150, the guide plate body 110 can be disassembled into a first guide plate 112 and a second guide plate 114. The first guide plate 112 has a connection part 116 protruding from the first guide plate 112 so that the first guide plate 112 can be embedded in the second guide plate 114 along the direction that parallels to the pair of long side surfaces 150 to form a complete guide plate body 110. The first guide plate 112, the second guide plate 114, and the connection part 116 are integrally formed by additive manufacturing.

Continuingly refer to FIG. 1, the pedicle screw implantation guide plate 100 also comprises a plurality of positioning holes 120, a longitudinal connection channel 130 and an angle mark 118.

The longitudinal connection channel 130 passes through an interior of the guide plate body 110 which adjacent to one of the long side surfaces 150 to pass through the first guide plate 112 and the second guide plate 114. The function of the longitudinal connection channel 130 is to provide a fixed connection point to firmly connect the first guide plate 112 and the second guide plate 114 together by inserting the Kirschner wires 140 to ensure that the first guide plate 112 and the second guide plate 114 maintaining immobility and alignment during surgery. It helps ensure the overall stability and structural integrity of the pedicle screw implantation guide plate 100. The design of the longitudinal connection channel 130 makes the surgical process smoother, and the surgeon can more easily operate the pedicle screw implantation guide plate 100, while reducing the risk of surgical failure caused by the displacement or instability of the pedicle screw implantation guide plate 100.

A plurality of positioning holes 120 passes through the pair of flat surfaces 170 of the guide plate body 110, the plurality of positioning holes 120 is used to insert Kirschner wires 140 to perform pedicle screw implantation surgery. Through these positioning holes 120, the surgeon can determine in advance the position and angle at which the Kirschner wires 140 should be inserted, so that the screws can be implanted more quickly and accurately during the operation. The plurality of positioning holes 120 is arranged in an array. In one embodiment, the positioning holes 120 are arranged in a 5*14 matrix holes, and different positioning holes 120 have different medial inclination angles. The positioning holes 120 comprise pedicle positioning holes 126, spinous process positioning holes 122, and positioning holes for an upper edge of the vertebral body 124.

The spine is composed of solid vertebrae, which are separated by soft and elastic soft tissues such as intervertebral discs and facet joints. The vertebral body is in the front, while the facet joints, lamina and spinous process are in the rear, jointly protecting spinal cord and nerves in the middle. The pedicles are located at the back of each vertebra, forming the vertebral foramen, and together with the lamina form the spinal canal, which houses and protects the spinal cord.

A plurality of spinous process positioning holes 122 is disposed in a central area of the guide plate body 110. These spinous process positioning holes 122 are used to insert the Kirschner wires 140 to drive the Kirschner wires 140 into the spinous processes of the patient. The surgeon can accurately guide the position and direction of the Kirschner wires 140 through these spinous process positioning holes 122, thereby ensuring the stability and fixation of the vertebrae. The spinous process positioning hole 122 has a caudal inclination angle of 8° to 10° and a medial inclination angle of 5° to 7°. This specific angle design helps ensure the correct positioning of the Kirschner wires 140 in the spinous process.

A plurality of pedicle positioning holes 126 is disposed on both sides of the central area of the guide plate body 110, respectively located on the first guide plate 112 or the second guide plate 114. These pedicle positioning holes are used to insert the Kirschner wires 140 to position the pedicles of the patient. The surgeon can accurately position and fix the vertebrae through these pedicle positioning holes 126 during surgery, thereby ensuring the accuracy and success of the surgery and reducing the risks and complications of the surgery. The pedicle positioning hole has a special medial inclination angle ranging from 4° to 20°. This angle is designed to adapt to different pedicle shapes. The range of the medial inclination angle provides the surgeon with flexibility when dealing with the anatomical variations of different patients, ensuring that the inserted Kirschner wires 140 can be firmly fixed in the pedicle, providing reliable support and stability for the operation.

A plurality of positioning holes for an upper edge of the vertebral body 124 is located at both ends of the longitudinal connection channel 130 to allow the Kirschner wire 140 to pass through the longitudinal connection channel 130 and position an upper edge of the vertebral body on the body surface of the patient. The surgeon can accurately position the specific structure of the upper edge of the vertebral body of the patient through these positioning holes for an upper edge of the vertebral body 124 to ensure the accuracy and success of the operation. In addition, the positioning hole for an upper edge of the vertebral body 124 can also connect the first guide plate 112 and the second guide plate 114 through inserting the Kirschner wires 140.

The angle marks 118 are disposed on the long side surfaces 150 and the short side surfaces 160 of the guide plate body 110 as visual reference points to help the surgeon accurately adjust the position of the guide plate body 110 during surgery while reducing the need for repeated use of the C-arm, reducing surgical time and risk of ionizing radiation. The angle mark 118 has scale lines, scale points or other visual marks for indicating the specific angle and position of the guide plate. For example, if there are scale lines on the angle mark 118, the scale lines should be marked with numbers or symbols to represent different angles.

The following is a specific description of the surgical operation steps by using the pedicle screw implantation guide plate 100 of the present invention:
1. Disposing angle marks 118: Disposing angle marks 118 on the long side surfaces 150 and the short side surfaces 160 of the guide plate body 110. The angle mark 118 has scale lines, scale points or other visual marks for indicating the specific angle and position of the guide plate.
2. Placing the pedicle screw implantation guide plate 100 on the patient's body surface, and initially align the spinal structures that need to be positioned, such as the spinous process of the vertebral body, the left and right pedicles, etc.
3. Adjusting the position of the pedicle screw implantation guide plate 100: The surgeon observes and refers to the angle mark 118, then gradually rotates or moves the pedicle screw implantation guide plate 100 according to the scale line so that the mark on the scale line gradually approaches the target angle. When the mark on the scale line is consistent with the target angle, stop adjusting the pedicle screw implantation guide 100.
4. Fixing the pedicle screw implantation guide plate 100: Once the position of the pedicle screw implantation guide plate 100 is determined, insert the Kirschner wires 140 into the pedicle positioning holes 126 and the spinous process positioning holes 122 to fix the pedicle screw implantation guide plate 100 at the position of the pedicle and spinous process.
5. Driving the screws: Removing the pedicle screw implantation guide plate 100 and driving the screws into the pedicle and spinous process along the Kirschner wires 140.

It should also be noted that the terms “comprising”, “including” or any other variation thereof are intended to cover a non-exclusive inclusion, such that a process, method, good or device that includes a list of elements not only includes those elements, but also includes other elements not expressly listed or that are inherent to the process, method, article, or equipment. Without further limitation, an element defined by the statement “comprises a . . . ” does not exclude the presence of additional identical elements in a process, method, article, or apparatus that includes the stated element.

It should be understood that the examples and embodiments described herein are for illustrative purposes only, and that various modifications or changes thereto will be suggested to those skilled persons in the art and will be included within the spirit and scope of the present invention as well as the within the scope of the claims.

1. A pedicle screw implantation guide plate, comprising:
a guide plate body with an opposite pair of long side surfaces, an opposite pair of short side surfaces and an opposite pair of flat surfaces, wherein the guide plate body comprises a first guide plate and a second guide plate, the first guide plate has a connection part protruding from the first guide plate, and the first guide plate is embedded in the second guide plate through the connection part along a direction that parallels to the pair of long side surfaces to form the guide plate body;
a longitudinal connection channel passed through an interior of the guide plate body which adjacent to one of the pair of long side surfaces to pass through the first guide plate and the second guide plate, the longitudinal connection channel is used to insert a first Kirschner wire to connect the first guide plate and the second guide plate;
a plurality of positioning holes passed through the pair of flat surfaces and the pair of short side surfaces; and
an angle mark disposed on the pair of long side surfaces and the pair of short side surfaces of the guide plate body for serving as a visual reference point to adjust a position of the guide plate body.

2. The pedicle screw implantation guide plate of claim 1, wherein the plurality of positioning holes is arranged in an array.

3. The pedicle screw implantation guide plate of claim 1, wherein the plurality of positioning holes comprises:
a plurality of spinous process positioning holes is disposed in a central area of the guide plate body for inserting a part of a plurality of second Kirschner wires to drive into the spinous process in an individual body, wherein the central area comprises an overlapping portion of the connection part and the second guide plate and a portion of the first guide plate adjacent to the connection part;
a plurality of pedicle positioning holes is disposed on the first guide plate and the second guide plate at both sides of the central area for inserting another part of the second Kirschner wires to locate the pedicles in the individual body; and
a plurality of positioning holes for an upper edge of the vertebral body is located at both ends of the longitudinal connection channel to allow the first Kirschner wire to pass through the longitudinal connection channel and position an upper edge of the vertebral body on a body surface of the individual body.

4. The pedicle screw implantation guide plate of claim 3, wherein the plurality of pedicle positioning holes have a medial inclination angle of 4° to 20°.

5. The pedicle screw implantation guide plate of claim 3, wherein the plurality of spinous process positioning holes have a caudal inclination angle of 8° to 10° and a medial inclination angle of 50 to 7°.

6. The pedicle screw implantation guide plate of claim 1, wherein the first guide plate and the second guide plate respectively have an integrally formed by additive manufacturing.
